(19) United States
(12) Patent Application Publication
(10) Pub. No.: US 2004/0037810 A1
(43) Pub. Date: Feb. 26, 2004

(54) Biomaterials comprised of preadipocyte cells for soft tissue repair

(76) Inventors: Dennis Von Heimburg, Aachen (DE); Alessandra Pavesio, Padova (IT)

Correspondence Address: BIRCH STEWART KOLASCH & BIRCH, PO BOX 747, FALLS CHURCH, VA, 22040-0747, US

(21) Appl. No.: 10/275,085

(22) PCT Filed: May 3, 2001
(86) PCT No.: PCT/EP01/05087

(51) Int. Cl. A61K 45/00; C08B 37/00

(52) U.S. Cl. 424/93.7; 536/53

(57) ABSTRACT

The present invention is directed to the use of support materials and injectable preparations comprised of esters, especially benzyl esters, and amides of hyaluronic acid in reconstructive surgery for soft tissue, particularly support materials and injectable preparations for adipose precursor cells.

### SUMMARY OF THE INVENTION

The present invention is directed to the use of support materials and injectable preparations comprised of esters, especially benzyl esters, and amides of hyaluronic acid in reconstructive surgery for soft tissue, particularly support materials and injectable preparations for adipose precursor cells.

### BACKGROUND OF THE INVENTION

The correction of soft tissue defects is an important challenge in plastic and reconstructive surgery. Adipose tissue as a free graft has been used for the reconstruction of soft tissue defects for more than 100 years. But there has been a lack of an optimal implant material for soft tissue replacement. Free adipose tissue grafts are used but the results are poor and unpredictable. The transplants are largely absorbed and replaced by fibrous tissue and oil cysts. The recently revived technique of injecting aspirated fat fragments also gives unsatisfactory results, ranging from 50% shrinkage of the graft to complete resorption. The poor results of free fat autotransplantation are thought to be due to the low tolerance of the fat cells to ischemia and the slow rate of revascularisation.

Adipose precursor cells located in the stroma of adipose tissue can be isolated and cultured. These cells demonstrate in vitro differentiation and dedifferentiation under different conditions and are a possible source for soft tissue engineering because of the ability to proliferate and differentiate. In a preliminary study we observed that preadipocytes revascularise rapidly and reaccumulate fat after transplantation. In a recent study rat preadipocytes differentiated in PLGA scaffolds after grafting.

Recently, mesenchymal stem cells (MSCs), obtained from adult bone marrow, have been used for the production of various tissue cell types. These isolated stem cells are not totipotent, as are embryonic stem cells, but pluripotent and capable of differentiating into connective tissue and its derivatives. Mesenchyme is a source not only of connective tissue such as muscle, tendon and ligament, but also blood, cartilage, bone, fat cells and the outer layers of blood vessels. To date, MSCs have been successfully differentiated into adipose cells, chrondrocyte cells and osteocyte cells (Pittenger et al. (1999) Science 284:143-7).

But there is still a need for a good bioartificial soft tissue filler for tissue engineering, one that ideally would be a delivery vehicle to support human preadipocytes in grafting procedures. The material should provide a structure for supporting implanted cells (pre-adipose cells) and a structure that allows the cells to invade and differentiate after transplantation. Introduction of endothelial cells, which are angiogenic, would also enhance the performance of bioartificial soft tissue filler material. A higher number of mature adipocytes are found in well vascularized adipose tissue, possibly due to the influence that endothelial cells have on differentiating preadipocytes and adipocytes. Consequently, endothelial cells are particularly useful in adipose tissue engineering that is based on preadipocytes. Mechanical stability of the carrier is also important and the material/carrier may not be resorbed too quickly after transplantation.

These needs are met by the present invention which provides an optimal matrix for isolated and cultured human preadipocytes, mesenchymal stem cells and endothelial cells in vitro and in vivo. The present invention is also useful as a bioartificial soft tissue filler material, particularly as a scaffold for preadipocytes, mesenchymal stem cells and/or endothelial cells with the ability to support in vivo adipogenesis.

### BRIEF DESCRIPTION OF THE FIGURES

FIG. 1 Human preadipocytes 24 hours after being seeded on HYAFF 11 sponge (HS+). Good adherence of viable cells to the scaffold can be observed. Cytoplasmic vacuoles are seen and these store lipid as typical morphological signs of differentiation. (toluidine-blue, sc=scaffold).

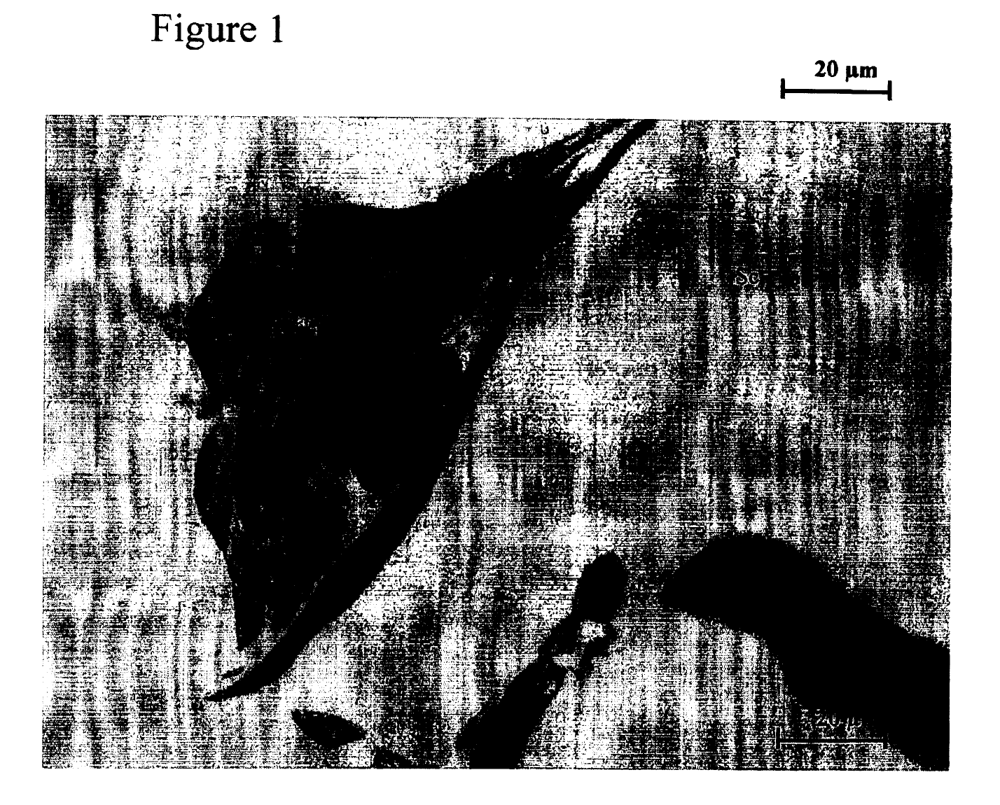

FIG. 2 Macroscopic appearance of explanted HS grafts after 3 weeks in the nude mouse. A thin yellow tissue presented on the preadipocyte grafts with new vessel formation (right). The contralateral control sponge out of the same animal revealed almost no change to the sponge and no vessels (left).

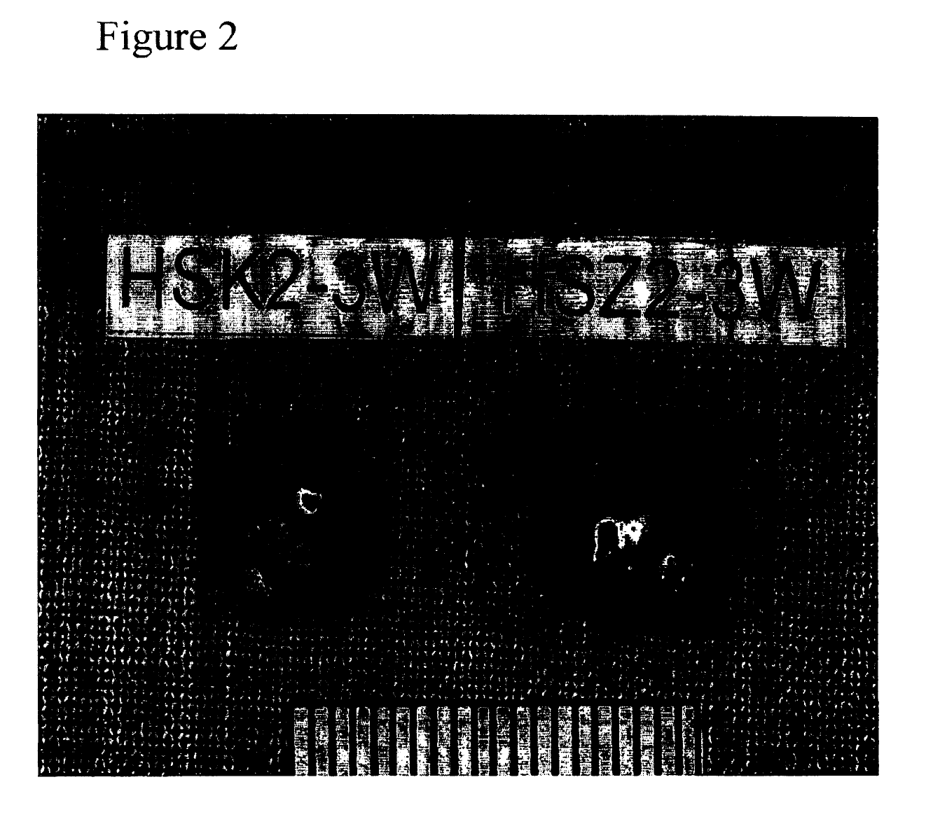

FIG. 3 Microscopical view of the HS+ section after 3 weeks in the nude mouse. Differentiated clusters of adipocytes are more numerous in open pores even in the centre of the sponge. Note the intense red stain of lipid containing mature adipocytes and the scaffold structure. (oil-red, sc=scaffold)

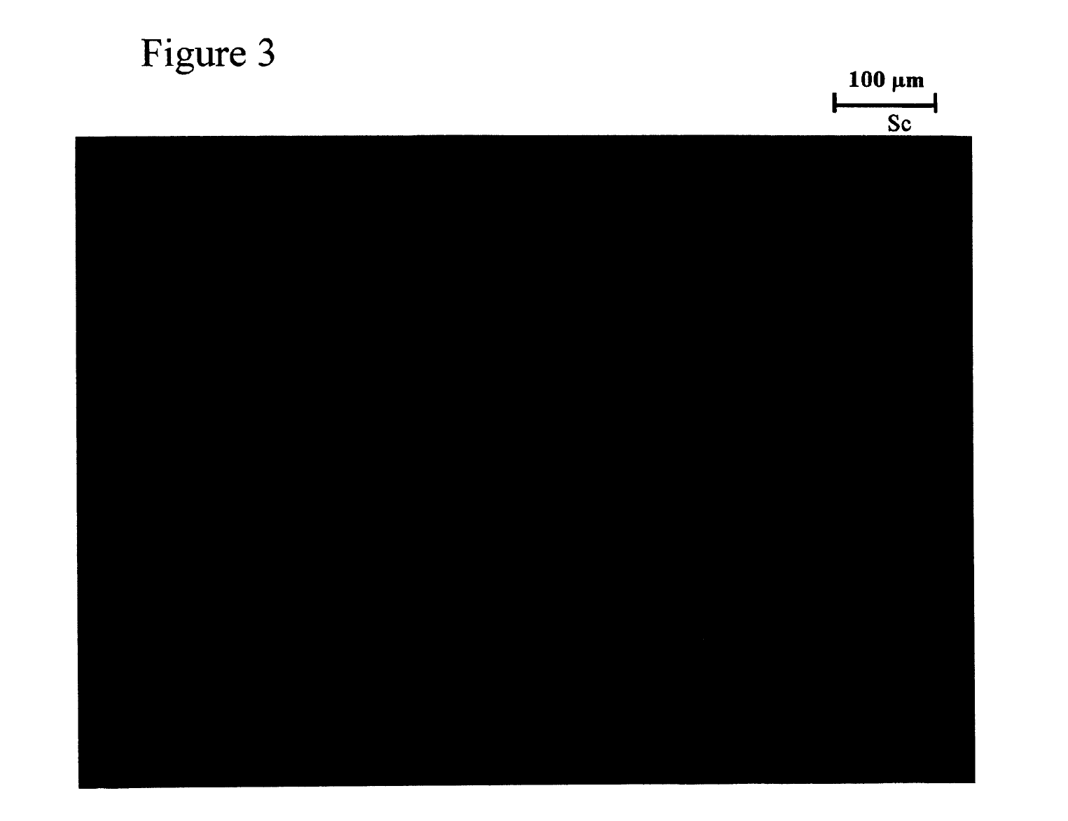

FIG. 4 Invaded human cells in preadipocyte/scaffold grafts HS+ in group A (3 weeks in vivo). Note good and homogenous distribution of human cells in the scaffold. (mah-vim stain, sc=scaffold)

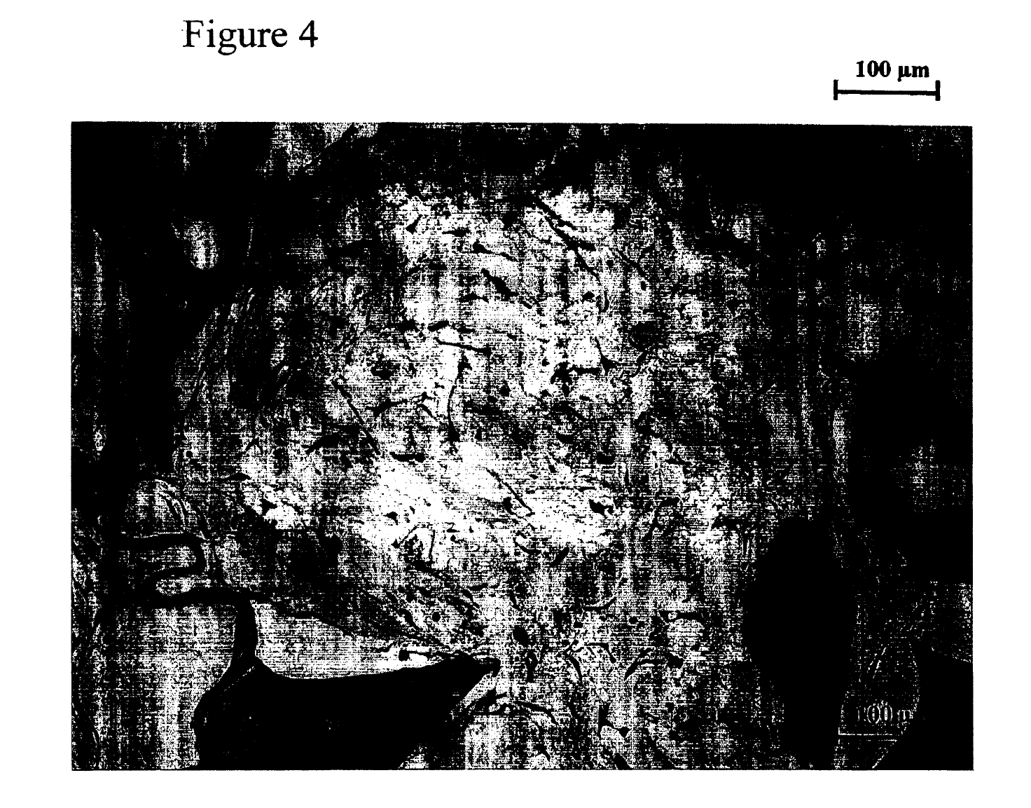

FIG. 5 Cellularity of donor and host cells in preadipocyte/scaffold constructs and controls.

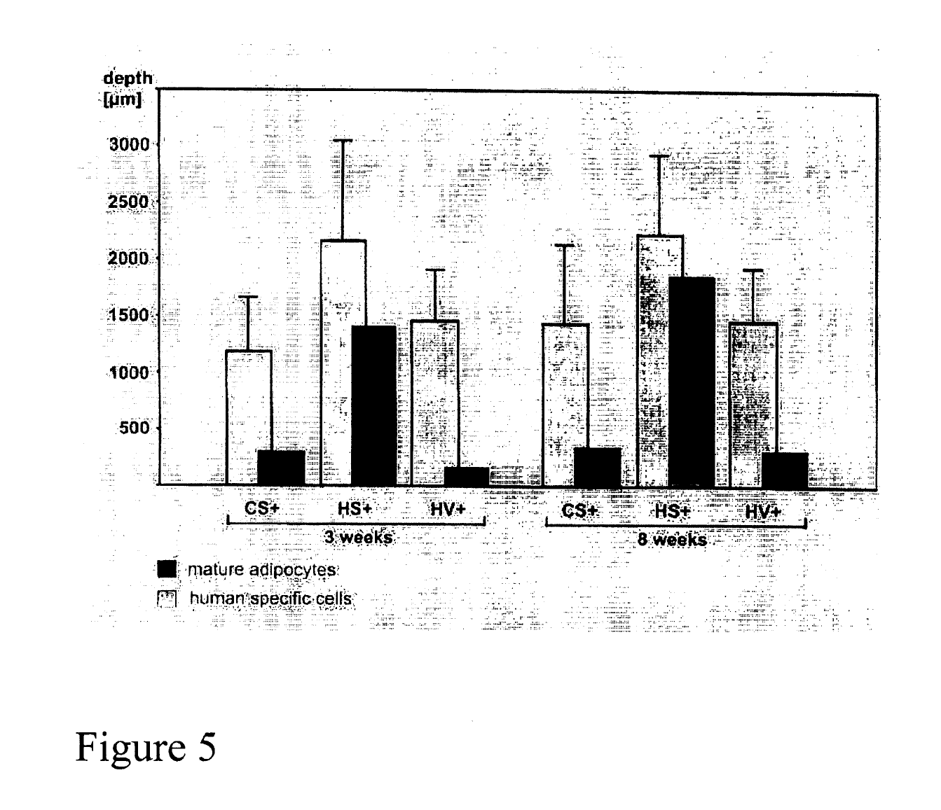

FIG. 6 Ultrastructure of preadipocytes in the nonwoven matrix after 3 weeks in vivo. The cells contain multiple cytoplasmic lipid droplets. The fibers and preadipocytes are closely packed together. Note the HYAFF 11 fiber in the left above corner and the new ECM inbetween.

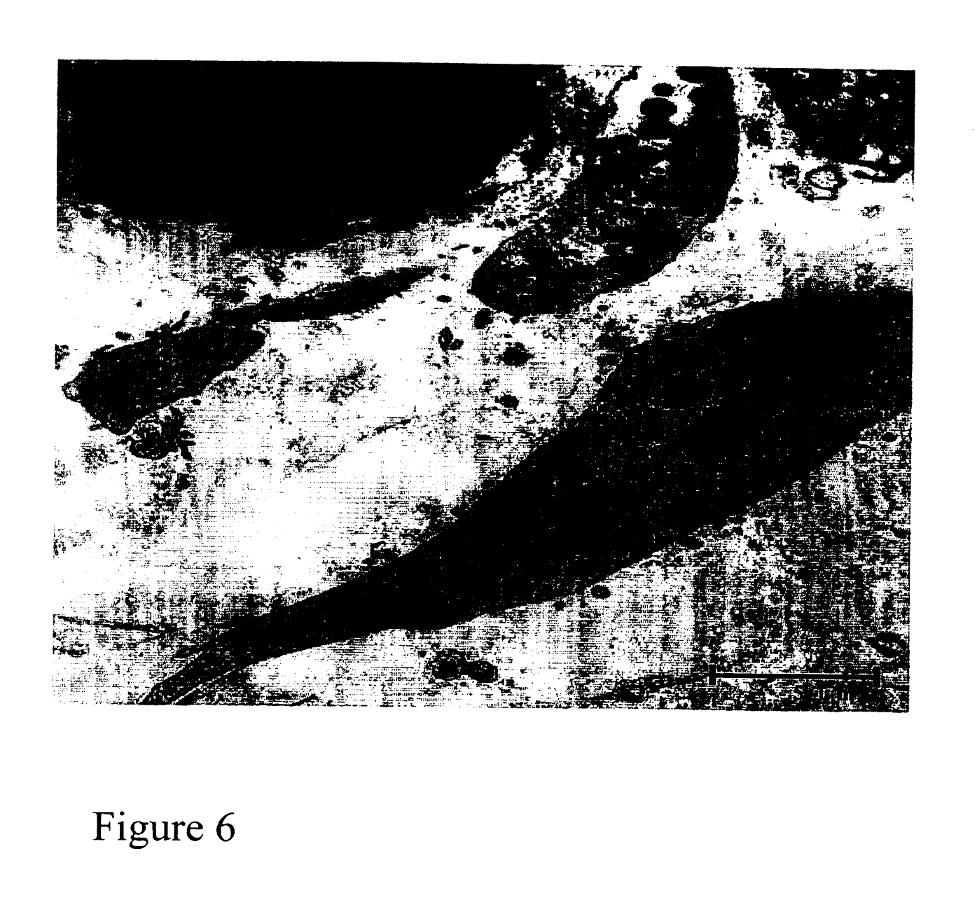

FIG. 7 Differentiated adipocytes in a cluster in HYAFF 11 sponge after 8 weeks in vivo. The cells contain single lipid droplets of large sizes (>50 μm) and show typical signet ring appearance. Note the new collagen fibers in between the cells.

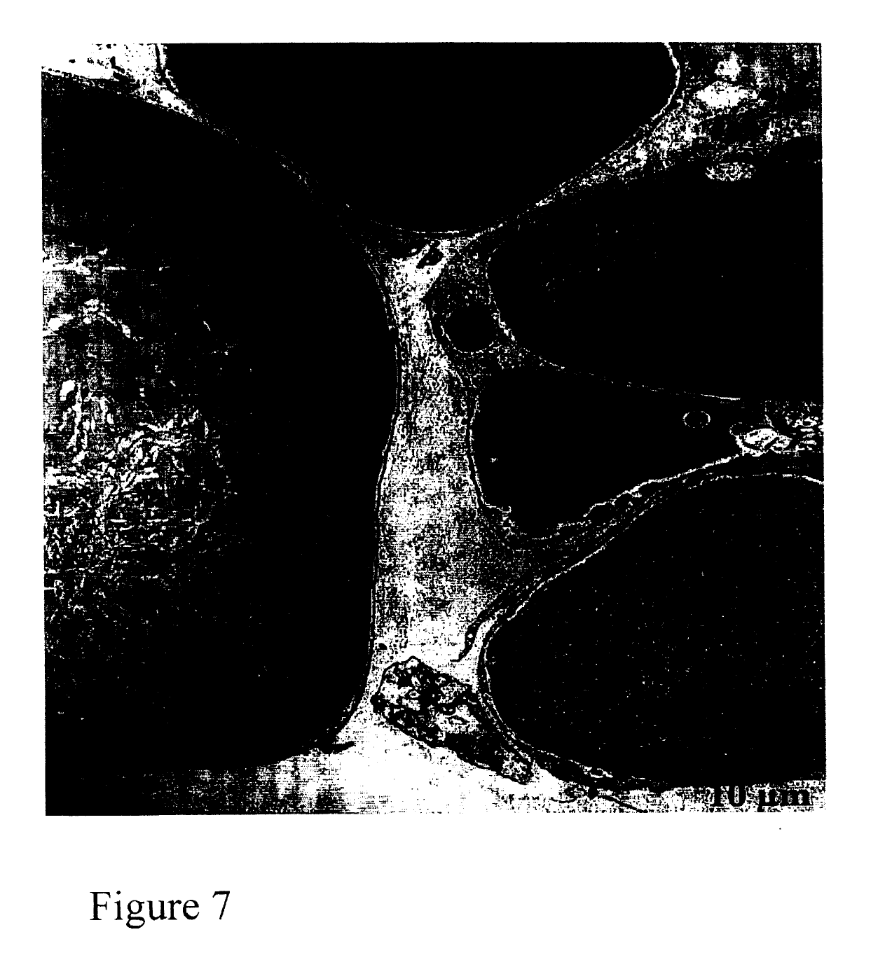

### DETAILED DESCRIPTION OF THE INVENTION

Soft tissue defect correction by plastic or reconstructive surgery can be performed by implantation of isolated and culture-expanded adipose precursor cells, MSCs and/or endothelial cells. Adipose precursor cells, when implanted, differentiate into adipocytes, which are animal connective tissue cells that are specialized for the synthesis and storage of fat. Although MSCs may, in some cases, first require in vitro manipulation to initiate differentiation, these cells are also capable of producing adipocytes. But appropriate supports or scaffolds are needed in this soft tissue engineering to allow and encourage differentiation and proliferation of the precursor cells, MSCs or endothelial cells.

In the present invention, the biomaterial for soft tissue repair is comprised of a preferred support or scaffold comprised of a benzyl ester of hyaluronic acid in the form of a sponge or non-woven material. Alternatively, the biomaterial is an injectable preparation comprised of a total water-soluble hyaluronic acid derivative or a partially water-soluble hyaluronic acid derivative, wherein the derivatives are particularly a benzyl ester or an amide derivative. In particular, benzyl ester derivatives with 85% or less esterification and the dodecylamide of HA are preferred for injectable preparations. The preparation of such benzyl esters is described in EP 0 216 453 B1 and the preparation of amide derivatives is described in WO 00/01733.

The term “hyaluronic acid” (also referred to as “HA” hereinafter) is used in literature to designate an acidic polysaccharide with various molecular weights constituted by residues of D-glucoronic acid and N-acetyl-D-glucosamine, which naturally occur in cellular surfaces, in the basic extracellular substances of the connective tissue of vertebrates, in the synovial fluid of joints, in the vitreous humor of the eye, in the tissue of the human umbilical cord and in cocks' combs.

Hyaluronic acid plays an important role in an organism, firstly as mechanical support of the cells of many tissues, such as the skin, the tendons, muscles and cartilage and it is, therefore, the main component of the intracellular matrix. But hyaluronic acid also performs other functions in the biological processes, such as the hydration of tissues, lubrication, cellular migration, cell function and differentiation. (See for example A. Balazs et al., Cosmetics & Toiletries, No. 5/84, pages 8-17). Hyaluronic acid may be extracted from the above mentioned natural tissues, such as cocks' combs, or also from certain bacteria. Today, hyaluronic acid may also be prepared by microbiological methods. The molecular weight of whole hyaluronic acid obtained by extraction is in the region of 8-13 million. However, the molecular chain of the polysaccharide can be degraded quite easily under the influence of various physical and chemical factors, such as mechanical influences or under the influence of radiation, hydrolyzing, oxidizing or enzymatic agents. For this reason often in the ordinary purification procedures or original extracts, degraded fractions with a lower molecular weight are obtained. (See Balazs et al. cited above). Hyaluronic acid, its molecular fractions and the respective salts have been used as medicaments and their use is also proposed in cosmetics (see for example the above mentioned article by Balazs et al. and French Patent No. 2478468).

As a therapeutic agent, hyaluronic acid and its salts have been used especially in therapy for arthropathies, such as in veterinary medicine for the cure of arthritis in horses [Acta Vet. Scand. 167, 379 (1976)]. As an auxiliary and substitutional therapeutic agent for natural tissues and organs, hyaluronic acid and its molecular fractions and their salts have been used in ophthalmic surgery (see for example Balazs et al., Modern Problems in Ophthalmology, Vol. 10,1970, p. 3—E. B. Strieff, S. Karger eds., Basel; Viscosurgery and the Use of Sodium Hyaluronate During Intraocular Lens Implantation, Paper presented at the International Congress and First Film Festival on Intaocular Implantation, Cannes, 1979; U.S. Pat. No. 4,328,803 with a summary of the literature on the uses of HY in ophthalmology; and U.S. Pat. No. 4,141,973.) European patent publication no. 0138572 describes a molecular fraction of hyaluronic acid which can be used, for example, as sodium salt for intraocular and intraarticular injections suitable for the substitution of internal fluids of the eye and in arthropathy therapies.

Hyaluronic acid may also be used as an additive for a wide variety of polymeric materials used for medical and surgical articles, such as polyurethanes, polyesters, polyolefins, polyamides, polysiloxanes, vinylic and acrylic polymers and carbon fibers with the effect of rendering these materials biocompatible. In this case the addition of HY or one of its salts is effected for example by covering the surface of such materials, by dispersion in the same or by both of these procedures. Such materials may be used for the manufacture of various sanitary and medical articles, such as cardiac valves, intraocular lenses, vascular clips, pacemakers and such (see U.S. Pat. No. 4,500,676).

Although the term “hyaluronic acid” is commonly used in an improper sense, meaning, as can be seen from above, a whole series of polysaccharides with alternations of residues of D-glucuronic acid and N-acetyl-D-glucosamine with varying molecular weights or even degraded fractions of the same, and although the plural form “hyaluronic acids” may seem more appropriate, the discussion herein shall continue to use the singular form to refer to hyaluronic acid in its various forms including its molecular fractions, and the abbreviation “HA” will also often be used to describe this collective term.

EP 0 216 453 B1 describes total or partial esters of hyaluronic acid with an alcohol of the aliphatic, or araliphatic series or a salt of such partial ester with an inorganic or organic base. Such esters possess interesting bio-plastic and pharmaceutical properties and may be used in various fields, including cosmetics, surgery and medicine. In the case of hyaluronic acid, in which the new products qualitatively possess the same or similar physical-chemical, pharmacological and therapeutic properties, they are considerably more stable, especially regarding the action of the natural enzymes responsible for the degradation of the polysaccharaide molecule in the organism, such as especially hyaluronidase, and they, therefore, conserve the above mentioned properties for very long periods.

WO 00/01733 describes amides of hyaluronic acid and derivatives thereof obtained by reacting the carboxy groups or amino groups originating from deacetylation reactions with amines, and acids of the aliphatic, aromatic, arylaliphatic, cycloaliphatic, heterocyclic series, and without the use of spacer chains. These compounds can be either water soluble or insoluble, according to the acid, the amine, the percentage of amide bond or the derivative of hyaluronic acid used to prepare the amide. These amides can be used in various fields of surgery, in the prevention of post-surgical adhesions and hypertrophic scarring, cardiology, dermatology, opthalmology, otorhinolaryngology, dentistry, orthopaedics, gyaecology, urology, extra-corporeal blood circulation and oxygenation, cosmetics and angiology. Like the esters described above, these amides of hyaluronic acid retain the viscosity of free hyaluronic acid, but are more stable and persist longer before being degraded.

In the present invention, the ester of HA with benzyl alcohol (the benzyl ester) or an amide of HA is utilized in the support or scaffold for adipose precursor cells. The benzyl ester of HA utilized in the invention is preferably either a “total ester” (that is, a derivative wherein all of the carboxyl groups of the HA are esterified with benzyl alcohol) or a 5-99% ester (that is, a derivative wherein 5 to 99% of the carboxyl groups are esterified and the remaining groups salified). These derivatives provide preferred scaffold support materials for cultivating and growth of human preadipocytes, MSCs and/or endothelial cells. These derivatives can also be delivered with accompanying populations of cells (preadipocytes, mesenchymal stem cells or endothelial cells) by injection. Here, for example, a benzyl ester or an amide of HA is mixed with a population of preadipocytes and/or MSCs and/or endothelial cells and then injected into a site of soft tissue containing a depression, defect, wrinkle or deformity. Hyaluronic acid derivatives, especially those wherein 85% or less of the carboxyl groups of the HA are esterified with benzyl alcohol and the dodecyl amide of HA, are particularly preferred. One especially preferred combination is the dodecyl amide of HA and preadipocyte cells.

The benzyl esters, as noted above, can be prepared according to the procedures described in EP 0 216 453 B1 (see Examples 1-4). The amides can be prepared according to the procedures described in WO 00/01733 (see Examples 5-24.

### EXAMPLE 1

### Preparation of the Benzylester of Hyaluronic Acid (HY)

12.4 g of HY tetrabutylammonium salt with a molecular weight of 170,000 corresponding to 20 m.Eq. of a monomeric unit are solubilized in 620 ml of dimethylsulfoxide at 25°, 4.5 g (25 m.Eq.) of benzyl bromide and 0.2 g of tetrabutylammonium iodide are added, the solution is kept for 12 hours at 30°.

The resulting mixture is slowly poured into 3,500 ml of ethyl acetate under constant agitation. A precipitate is formed which is filtered and washed four times with 500 ml of ethyl acetate and finally vacuum dried for twenty four hours at 30°.

9 g of the benzyl ester product in the title are obtained. Quantitative determination of the ester groups is carried out according to the method described on pages 169-172 of Siggia S. and Hanna J. G. “Quantitative organic analysis via functional groups” 4^{th} edition, John Wiley and Sons.

### EXAMPLE 2

### Preparation of the Benzyl Ester of Hyaluronic Acid HY

3 g of the potassium salt of HY with a molecular weight of 162,000 are suspended in 200 ml of dimethylsulfoxide; 120 mg of tetrabutylammonium iodide and 2.4 g of benzyl bromide are added.

The suspension is kept in agitation for 48 hours at 30°. The resulting mixture is slowly poured into 1,000 ml of ethyl acetate under constant agitation. A precipitate is formed which is filtered and washed four times with 150 ml of ethyl acetate and finally vacuum dried for twenty four hours at 30°.

3.1 g of the benzyl ester product in the title are obtained. Quantitative determination of the ester groups is carried out according to the method described on pages 169-172 of Siggia S. and Hanna J. G. “Quantitative organic analysis via functional groups” 4^{th} edition, John Wiley and Sons.

The scaffold support material is comprised of a spongy material comprised of the HA benzyl ester, and can be prepared as follows.

### EXAMPLE 3

### Preparation of a Spongy Material Made with Hyaluronic Acid Esters

1 g of benzyl esters of hyaluronic acid with a molecular weight of 170,000 in which all the carboxylic groups are esterified (obtained for example as described above) are dissolved in 5 ml of dimehtylsulfoxide. To each 10 ml of solution prepared, a mixture of 31.5 g of sodium chloride with a degree of granularity corresponding to 300μ, 1.28 g of sodium bicarbonate and 1 g of citric acid is added and the whole is homogenized in a mixer.

The pasty mixture is stratified in various ways, for instance by means of a mange consisting of two rollers which turn opposite each other at an adjustable distance between the two. Regulating this distance the past is passed between the rollers together with a strip of silicone paper which acts as a support to the layer of paste thus formed. The layer is cut to the desired dimensions of length and breadth, removed from the silicone, wrapped in filter paper and emerged in a suitable solvent, such as water. The sponges thus obtained are washed with a suitable solvent, such as water, and optionally sterilized with gamma rays.

A scaffold support comprised of a non-woven material of the HA benzyl ester (also known as HYAFF 11) can be prepared as described in U.S. Pat. No. 5,520,916 according to the following procedures.

### EXAMPLE 4

A solution of HYAFF 11 in dimethylsulfoxide at a concentration of 135 mg/ml is prepared in a tank and fed by a gear metering pump into a spinneret for wet extrusion composed of 3000 holes each measuring 65 microns.

The extruded mass of threads passes into a coagulation bath containing absolute ethanol. It is then moved over transporting rollers into two successive rinsing baths containing absolute ethanol. The drafting ratio of the first roller is set at zero while the drafting ratio between the other rollers is set at 1.05. Once it has been passed through the rinsing baths, the hank of threads is blown dry with hot air at 45.degree.-50.degree. C. and cut with a roller cutter into 40 mm fibers.

The mass of fibers thus obtained is tipped into a chute leading to a carding/cross lapping machine from which it emerges as a web, 1 mm thick and weighing 40 mg/mq. The web is then sprayed with a solution of HYAFF 11 in dimethylsulfoxide at 80 mg/ml, placed in an ethanol coagulation bath, in a rinsing chamber, and lastly in a drying chamber.

The final thickness of the material is 0.5 mm.

### EXAMPLE 5

### Preparation of Partially N-deacetylated Hyaluronic Acid in the Form of Sodium Salt (DHA/Na)

One gram of sodium hyaluronate, with a mean molecular weight of 600 Kda, is solubilized in 50 ml of a 1% solution of hydrazine sulphate in hydrazine monohydrate. This is left to react under agitation for five days (120 hours) at 55° C., after which the reaction is stopped by adding 100 ml of ethanol. The precipitate thus formed is filtered through a Gooch crucible, washed with ethanol and then dried at room temperature at reduced pressure. Any hydrazide of hyaluronic acid that will probably be formed during the reaction with hydrazinolysis is destroyed by reaction with HIO₃ (iodic acid). As the reaction may be very vigourous, it is conducted while cooling the reaction container in iced water. The product of hyrazinolysis is solubilized in 50 ml of a solution of 5% sodium acetate and reacted with 25 ml of a 0.5 M solution of iodic acid. The reaction proceeds for 30 minutes under agitation, after which 5 ml of a 57% solution of HI is added to destroy any unreacted HIO₃. The iodine that has formed is extracted from the aqueous solution with at least three 30-ml aliquots of ethyl ether (until complete decoloring of the aqueous phase). The aqueous solution is brought to neutral pH by adding a solution of NaOH 0.5 M followed by treatment with 100 ml of ethanol. The precipitate obtained is filtered with a Gooch cricible, washed with ethanol and then dried at room temperature and at reduced pressure. The product obtained is characterized analytically to determine the percentage of N-deacetylated groups and the mean molecular weight.

| | |
|---|---|
| Yield of the reaction | 90% |
| % of N-deacetylation | 26% |
| mean molecular weight | 130 Kda |

### EXAMPLE 6

### Preparation of the Salt of Hyaluronic Acid Partially N-deacetylated with Tetrabutylammonium (DHA/TBA)

One gram (2.5 mmol.) of hyaluronic acid sodium salt, partially N-deacetylated, is solubilized in 60 ml of water and the solution is percolated through a column filled with 25 ml of a sulfonic resin in the form of tetrabutylammonium salt (TBA). The sulphonic resin in H⁺ form is activated with a 40% solution w/v of TBAOH. The eluate, containing N-deacetylated, hyaluronic acid TBA salt is collected and freeze-dried.

### EXAMPLE 7

### Preparation of p-NO₂-phenylester of Benzoic Acid (Acylating Agent)

Ten grams (0.082 mol.) of benzoic acid is solubilized in 800 ml of CH₂Cl₂, after which 11.4 g (0.082 mol.) of p-NO₂-phenol and 16.9 g (0.082 mol.) of DCC (Dicyclohexylcarbodiimide) are added. The reaction proceeds for 2 hours, while the solution is boiled and refluxed. Subsequently, the dicyclohexylurea that forms is filtered and the filtered product is dried with a rotavapor under reduced pressure. The product thus obtained is purified by repeated crystallization in ethyl acetate. The crystals are filtered and placed to dry at room temperature at reduced pressure. The derivative is characterized by TLC analysis (eluent: CH₂Cl₂/ethyl acetate 90/10 and Rf=0.77) and by IR and UV spectroscopy.

| | |
|---|---|
| Yield of the reaction | 92% |

### EXAMPLE 8

### Preparation of p-NO₂-phenylester of Cinnamic Acid (Acylating Agent)

Twelve grams (0.082 mol.) of cinnamic acid is solubilized in 800 ml of CH₂Cl₂, after which 11.4 g (0.082 mol.) of p-NO₂-phenol and 16.9 g (0.082 mol.) of DCC (Dicyclohexylcarbodiimide) are added. The reaction proceeds for 2 hours, while the solution is boiled and refluxed. Subsequently, the dicyclohexylurea that forms is filtered and the filtered product is dried with a rotavapor under reduced pressure. The product thus obtained is purified by repeated crystallization in ethyl acetate. The crystals are filtered and placed to dry at room temperature at reduced pressure. The derivative is characterized by TLC analysis (eluent: CH₂Cl₂/ethyl acetate 90/10 and Rf=0.77) and by IR and UV spectroscopy.

| | |
|---|---|
| Yield of the reaction | 89% |

### EXAMPLE 9

### Preparation of p-NO₂-phenylester of Dodecanoic Acid (Acylating Agent)

Sixteen grams (0.082 mol.) of dodecanoic acid is solubilized in 1 liter of CH₂Cl₂, after which 11.4 g (0.082 mol.) of p-NO₂-phenol and 16.9 g (0.082 mol.) of DCC (Dicyclohexylcarbodiimide) are added. The reaction proceeds for 2 hours, while the solution is boiled and refluxed. Subsequently, the dicyclohexylurea that forms is filtered and the filtered product is dried with a rotavapor under reduced pressure. The product thus obtained is purified by repeated crystallization in ethyl acetate. The crystals are filtered and placed to dry at room temperature at reduced pressure. The derivative is characterized by TLC analysis (eluent: CH₂Cl₂/ethyl acetate 90/10 and Rf=0.77) and by IR and UV spectroscopy.

| | |
|---|---|
| Yield of the reaction | 93% |

### EXAMPLE 10

### Preparation of p-NO₂-phenylester of Stearic Acid (Acylating Agent)

23.3 grams of stearic acid is solubilized in 1 liter of CH₂Cl₂, after which 11.4 g (0.082 mol.) of p-NO₂-phenol and 16.9 g (0.082 mol.) of DCC (Dicyclohexylcarbodiimide) are added. The reaction proceeds for 2 hours, while the solution is boiled and refluxed. Subsequently, the dicyclohexylurea that forms is filtered and the filtered product is dried with a rotavapor under reduced pressure. The product thus obtained is purified by repeated crystallization in ethyl acetate. The crystals are filtered and placed to dry at room temperature at reduced pressure. The derivative is characterized by TLC analysis (eluent: CH₂Cl₂/ethyl acetate 90/10 and Rf=0.77) and by IR and UV spectroscopy.

| | |
|---|---|
| Yield of the reaction | 87% |

### EXAMPLE 11

### Preparation of p-NO₂-phenylester of o-acetyl Salicyclic Acid (Acylating Agent)

14.7 grams of acetylsalicyclic acid is solubilized in 1 liter of CH₂Cl₂, after which 11.4 g (0.082 mol.) of p-NO₂-phenol and 16.9 g (0.082 mol.) of DCC (Dicyclohexylcarbodiimide) are added. The reaction proceeds for 2 hours, while the solution is boiled and refluxed. Subsequently, the dicyclohexylurea that forms is filtered and the filtered product is dried with a rotavapor under reduced pressure. The product thus obtained is purified by repeated crystallization in ethyl acetate. The crystals are filtered and placed to dry at room temperature at reduced pressure. The derivative is characterized by TLC analysis (eluent: CH₂Cl₂/ethyl acetate 90/10 and Rf=0.77) and by IR and UV spectroscopy.

| | |
|---|---|
| Yield of the reaction | 80% |

### EXAMPLE 12

### Preparation of Partially N-acetylated Hyaluronic Acid (with the Benzoic Acid Derivative)

One gram (1.6 mmol.) of DHA/TBA (26% deacetylation) is solubilized in 50 ml of DMSO, after which 5 ml of a 10% solution of p-NO₂-phenylester of benzoic acid (prepared according to Example 7) in DMSO is added. The reaction proceeds for 24 hours, under agitation at room temperature, after which it is blocked by adding 2.5 ml of a saturated solution of NaCl. This is left to react for 30 minutes and then 100 ml of ethanol is slowly added. The precipitate thus obtained is filtered through a Gooch, washed with ethanol and ethyl ether and lastly dried at room temperature and at reduced pressure. The derivative is analyzed by TLC (after hydrolysis of the amide), colorimetric analysis of the percentage of free NH₂ groups and IR and UV spectroscopy.

| | |
|---|---|
| Yield of the reaction | 85% |
| % free NH₂ | 11% |
| % N-acylation | 15% |

### EXAMPLE 13

### Preparation of Partially N-acetylated Hyaluronic Acid (with the Cinnamic Acid Derivative)

One gram (1.6 mmol.) of DHA/TBA (26% deacetylation) is solubilized in 50 ml of DMSO, after which 5 ml of a 10% solution of p-NO₂-phenylester of cinnamic acid (prepared according to Example 8) in DMSO is added. The reaction proceeds for 24 hours, under agitation at room temperature, after which it is blocked by adding 2.5 ml of a saturated solution of NaCl. This is left to react for 30 minutes and then 100 ml of ethanol is slowly added. The precipitate thus obtained is filtered through a Gooch, washed with ethanol/water 9:1, ethyl ether and lastly dried at room temperature and at reduced pressure. The derivative is analyzed by TLC (after hydrolysis of the amide), colorimetric analysis of the percentage of free NH₂ groups and IR and UV spectroscopy.

| | |
|---|---|
| Yield of the reaction | 85% |
| % free NH₂ | 11% |
| % N-acylation | 15% |

### EXAMPLE 14

### Preparation of Partially N-acetylated Hyaluronic Acid (with the Dodecanoic Acid Derivative)

One gram (1.6 mmol.) of DHA/TBA (26% deacetylation) is solubilized in 50 ml of NMP, after which 5 ml of a 10% solution of p-NO₂-phenylester of dodecanoic acid (prepared according to Example 9) in NMP is added. The reaction proceeds for 24 hours, under agitation at room temperature, after which it is blocked by adding 2.5 ml of a saturated solution of NaCl. This is left to react for 30 minutes and then 100 ml of ethanol is slowly added. The precipitate thus obtained is filtered through a Gooch, washed with ethanol and ethyl ether and lastly dried at room temperature and at reduced pressure. The derivative is analyzed by TLC (after hydrolysis of the amide), calorimetric analysis of the percentage of free NH₂ groups and IR and UV spectroscopy.

| | |
|---|---|
| Yield of the reaction | 88% |
| % free NH₂ | 10% |
| % N-acylation | 16% |

### EXAMPLE 15

### Preparation of Partially N-acetylated Hyaluronic Acid (with the Stearic Acid Derivative)

One gram (1.6 mmol.) of DHA/TBA (26% deacetylation) is solubilized in 50 ml of NMP, after which 5 ml of a 10% solution of p-NO₂-phenylester of stearic acid (prepared according to Example 10) in NMP is added. The reaction proceeds for 24 hours, under agitation at room temperature, after which it is blocked by adding 2.5 ml of a saturated solution of NaCl. This is left to react for 30 minutes and then 100 ml of ethanol is slowly added. The precipitate thus obtained is filtered through a Gooch, washed with ethanol and ethyl ether and lastly dried at room temperature and at reduced pressure. The derivative is analyzed by TLC (after hydrolysis of the amide), colorimetric analysis of the percentage of free NH₂ groups and IR and UV spectroscopy.

| | |
|---|---|
| Yield of the reaction | 85% |
| % free NH₂ | 12% |
| % N-acylation | 14% |

### EXAMPLE 16

### Preparation of Partially N-acetylated Hyaluronic Acid (with the Acetyl Salicylic Acid Derivative)

One gram (1.6 mmol.) of DHA/TBA (26% deacetylation) is solubilized in 50 ml of NMP, after which 5 ml of a 10% solution of p-NO₂-phenylester of acetyl salicylic acid (prepared according to Example 11) in NMP is added. The reaction proceeds for 24 hours, under agitation at room temperature, after which it is blocked by adding 2.5 ml of a saturated solution of NaCl. This is left to react for 30 minutes and then 100 ml of ethanol is slowly added. The precipitate thus obtained is filtered through a Gooch, washed with ethanol and ethyl ether and lastly dried at room temperature and at reduced pressure. The derivative is analyzed by TLC (after hydrolysis of the amide), colorimetric analysis of the percentage of free NH₂ groups and IR and UV spectroscopy.

| | |
|---|---|
| Yield of the reaction | 90% |
| % free NH₂ | 10% |

1. A biomaterial for soft tissue reconstruction comprised of
(a) a support material comprised of a benzyl ester of hyaluronic acid; and
(b) preadipocyte cells seeded on said support material.

2. A biomaterial according to claim 1, further comprising at least one member of the group consisting of mesenchymal stem cells and endothelial cells.

3. A biomaterial according to claim 1 or 2, wherein said benzyl ester of hyaluronic acid is a 100% ester, wherein all of the carboxyl groups of said hyaluronic acid are esterified with a benzyl alcohol residue.

4. A biomaterial according to claim 1 or 2, wherein said benzyl ester of hyaluronic acid is a 5 to 99% ester, wherein 5 to 99% of the carboxyl groups of said hyaluronic acid are esterified with a benzyl alcohol residue and the remaining groups are salified.

5. A biomaterial according to any one of claims 1-4, wherein said support material is a scaffold in the form of a spongy or non-woven material.

6. A biomaterial according to any one of claims 1-5, wherein at least one member of said group of preadipocyte cells, mesenchymal stem cells and endothelial cells are human cells.

7. A biomaterial according to any one of claims 1-6 for treating soft tissue damage.

8. A biomaterial according to any one of claims 1-7 for reconstructive treatment of soft tissue damage.

9. An injectable preparation for filling soft tissue defects and depressions comprised of
(a) a totally water soluble hyaluronic acid derivative or a partially water soluble hyaluronic acid derivative and
(b) preadipocyte cells suspended in the preparation.

10. The injectable preparation according to claim 9, further comprising at least one member of the group consisting of mesenchymal stem cells and endothelial cells.

11. An injectable preparation according to claim 9 or 10, wherein the totally water-soluble hyaluronic acid derivative is an amide of hyaluronic acid.

12. An injectable preparation according to claim 9 or 10, wherein the totally water-soluble hyaluronic acid derivative is a dodecyl amide of hyaluronic acid.

13. An injectable preparation according to claim 9 or 10, wherein the partially water-soluble hyaluronic acid derivative is a 5 to 99% ester, wherein 5 to 99% of the carboxyl groups of said hyaluronic acid are esterified with a benzyl alcohol residue and the remaining groups are salified.

14. An injectable preparation according to claim 9 or 10, wherein the partially water-soluble hyaluronic acid derivative is an 85% ester, wherein 85% of the carboxyl groups of said hyaluronic acid are esterified with a benzyl alcohol residue and the remaining groups are salified.

15. An injectable preparation according to claim 9 or 10, wherein at least one member of said group of preadipocyte cells, mesenchymal stem cells and endothelial cells are human cells.

16. A preparation according to any one of claims 9-15 for filling soft tissue defects and depressions.

17. A preparation according to any one of claims 9-16 for filling soft tissue defects, depressions, wrinkles and deformities of a patient in need thereof.

18. Use of a biomaterial according to any one of claims 1-5 or an injectable preparation according to any one of claims 9-15 in the reconstructive treatment of soft tissue damage.
